(19) United States
(12) Patent Application Publication
(10) Pub. No.: US 2006/0105023 A1
(43) Pub. Date: May 18, 2006

(54) Treatment of neurological disorders with nicotine

(76) Inventor: Joseph R. Knight, Agoura Hills, CA (US)

Correspondence Address: CHRISTIE, PARKER & HALE, LLP, PO BOX 7068, PASADENA, CA, 91109-7068, US

(21) Appl. No.: 11/319,898

(22) Filed: Dec. 27, 2005

Related U.S. Application Data
(63) Continuation-in-part of application No. 10/497,375, filed on Jun. 3, 2004.
(60) Provisional application No. 60/337,790, filed on Dec. 10, 2001.
(60) Provisional application No. 60/372,385, filed on Apr. 15, 2002.

(51) Int. Cl. A61K 31/4439; A61K 47/00

(52) U.S. Cl. 424/439; 514/343

(57) ABSTRACT

Neurological disorders, such as migraine and addiction to smoking tobacco, are treated by the administration of an effective amount of nicotine, preferably in a water solution of nicotine processed to diminish the odor, taste, and smell of nicotine.

### CROSS-REFERENCE TO RELATED APPLICATIONS

This application is a continuation-in-part of U.S. patent application Ser. No. 10/497,375 filed Jun. 3, 2004, which is a National Phase Patent Application of International Application No. PCT/US02/38655, filed on Dec. 4, 2002, which claims priority of Provisional U.S. Patent Application No. 60/337,790, filed Dec. 10, 2001 and Provisional U.S. Patent Application No. 60/372,385, filed Apr. 15, 2002.

### FIELD OF THE INVENTION

This invention relates to the treatment of neurological disorders, such as migraine headaches and addiction to smoking, with nicotine, and to processing nicotine for use in such treatment.

### BACKGROUND OF THE INVENTION

With the wide understanding of the health hazards caused by cigarette smoking, many efforts have been made to produce products which eliminate or reduce the need to smoke. For example, U.S. Pat. No. 3,901,248 to Lichtneckert, et al., discloses a chewable smoking substitute composition which includes nicotine adsorbed on a cation exchange resin, which is incorporated in a chewing gum base. When chewed, nicotine is released to diminish the urge to smoke.

More recently, U.S. Pat. No. 6,211,194 to Westman et al., and U.S. Pat. No. 6,268,386 to Thompson disclose beverages which have nicotine dissolved in them, and are intended to provide the consumer with sufficient nicotine to suppress the urge to smoke.

A problem with these prior art products is that the nicotine in them imparts a harsh or unpleasant taste, which discourages many of those addicted to smoking from using the products. One form of this invention provides a beverage which contains an effective amount of processed nicotine, and which is free from the objectionable taste, smell or odor of nicotine.

I have also discovered that the symptoms of migraine headaches can be alleviated or prevented by the administration of nicotine. Migraine headaches are so severe and disruptive of the life of the afflicted that many sufferers of that ailment may be willing to use nicotine in safe doses in almost any form to get relief, regardless of any objectionable taste, odor or smell of nicotine. However, preferably the nicotine is administered to the migraine patient in the convenient and unobjectionable beverage provided by this invention.

### SUMMARY OF THE INVENTION

Migraine headaches are treated by the administration of effective amounts of nicotine, preferably in a water solution of nicotine processed as described below. However, nicotine in suitable doses and in other forms can also be administered in other forms, such as injection, tablet, gel cap, transdermal patch, or gum.

One embodiment of this invention provides a beverage which includes processed nicotine, and therefore does not have a discernable characteristic nicotine odor, taste or smell when consumed. In brief, the beverage is prepared by stirring a heated mixture of nicotine or a nicotine-containing substance and water for at least about one minute, and preferably for at least about 30 minutes, and thereafter filtering the mixture. In the preferred process, the nicotine or nicotine-containing substance is mixed with water pre-heated to a temperature above about 40° C., and preferably to about the boiling point. The mixture is stirred while heated to keep the nicotine dispersed in the water. Thereafter, the mixture is filtered.

The nicotine-containing substance is selected from a group consisting of tobacco alkaloids, which include substantially pure nicotine (which may be levo nicotine, dextro nicotine, or a racemic mixture of the two), and nicotine-like or related pharmacologically active compounds such as nor-nicotine, lobeline and the like, as well as the free-base substance nicotine and all pharmacologically acceptable salts of nicotine, including acid addition salts. Nicotine salts are useful and include nicotine hydrogen tartrate and nicotine bitartrate, as well as nicotine hydrochloride, nicotine dihydrochloride, nicotine sulfate, nicotine citrate, nicotine zinc chloride monohydrate and nicotine salicylate, either alone or in combination. “Nicotine” is used herein to include all the foregoing tobacco alkaloids and nicotine salts.

“Nicotine” also includes the solid complex of one or more tobacco alkaloid compounds bound to an ion exchange resin, or other polymer release system, particularly a cation exchanger. Examples of nicotine ion exchange resins are set forth in U.S. Pat. No. 3,901,248 to Lichtneckert et al., referred to above. That patent is incorporated herein in full. Nicotine polacrilex is a source of nicotine. Other sources include cured tobacco leaves and other plants which contains sufficient nicotine to be effective.

When nicotine polacrilex (a powder) is mixed with water, the material does not readily go into solution at room temperature, but instead produces a slurry. Heating and stirring the slurry causes the nicotine polacrilex to go into solution or at least become thoroughly dispersed. The solution is cooled, and much of the ion exchange resin solidifies to form a precipitate which settles out of the mixture, which is preferably cooled to about room temperature, and thereafter filtered. In one form of the invention, the mixture is passed through activated charcoal. Preferably, the cooled solution is also passed under pressure through a mechanical filter having a nominal pore size of about one micron. The processed mixture can also be filtered through an ionic filter, such as a semi-permeable membrane used in reverse osmosis processes.

The stirred heated mixture of nicotine and water described above can be at about the concentration of nicotine desired in the final product, say, between about 0.2 mg and about 10 mg per about 250 ml, of water, but this requires heating and filtering the entire volume of the product. Preferably, the nicotine is present in the mixture at a much higher concentration, say at least about 1% by weight, and preferably between about 8% and about 12% by weight. After this concentrated mixture is heated, stirred, and filtered as described above, it is mixed with purified water to provide a final product (beverage) with the desired concentration of nicotine processed in accordance with one aspect of this invention. Although the modification of the processed nicotine is not fully understood at present, it is believed that the stirring, heating and filtering steps remove some components and thereby make the nicotine less objectionable when formulated in the final beverage product.

### DETAILED DESCRIPTION OF THE INVENTION

Nicotine in any suitable form, such as pure nicotine, tobacco leaves, nicotine alkaloids, or the various other sources of nicotine mentioned above, is mixed with water heated to at least about 40° C., and preferably to about the boiling point, and stirred vigorously for about one to about thirty minutes. Stirring of the mixture is stopped, permitting solids present to settle out. The mixture is thereafter filtered. If required, the filtrate is mixed with an appropriate amount of purified water to produce a beverage with the desired concentration of nicotine processed in accordance with one aspect of this invention.

In one embodiment of the invention, the nicotine is in the form of nicotine polacrilex in which nicotine is bound to an ion-exchange resin, as described in U.S. Pat. No. 3,901,248 referred to above. Nicotine polacrilex in powder form is commercially available from Spectrum Chemical Mfg. Corp. in Gardena, Calif. 90248. Twenty-five grams of nicotine polacrilex (15%, U.S.P.) was mixed in three gallons of water to form a slurry, which was heated to the boiling point (about 99° C.) while stirring for five to ten minutes. The nicotine polacrilex powder appeared to dissolve, or at least liquefy, so that it was uniformly dispersed in the mixture. Thereafter, the mixture was allowed to stand and cool to about room temperature. During the cooling process, a precipitate formed and settled to the bottom of the mixture. Supernatant liquid, which had a brownish color was taken from the mixture and passed through a 0.2 μm polyethersulfone (PES) membrane filter, and then through a medical grade granular activated carbon filter, producing a water-white filtrate, which was tasteless, odorless and colorless.

The PES membrane filter is available from PTI Advanced Filtration Inc. in Oxnard, Calif. 93030. The activated carbon filter is available from ResinTech Inc., in Cherry Hill, N.J. 08034 1409.

The filtered product can be consumed as drinking water, and has proved useful in suppressing the urge to smoke cigarettes. For example, many smokers have suppressed the urge to smoke by drinking between about 250 and about 500 ml of the treated water when experiencing the urge to smoke.

The presently preferred process for preparing a beverage with treated nicotine in accordance with this invention includes adding about one part pure liquid nicotine by volume to nine volumes of water which is pre-heated to a temperature of about 98° C. The nicotine can be acquired from Siegfried Limited in Zofingen, Switzerland under product number U1654. The mixture of nicotine and water is maintained at a temperature of about 98° C. and stirred for between about one and about thirty minutes. The mixture is cooled to a temperature less than about 50° C., and filtered through a commercially available filter having a pore size of about one micron. The filter can be made of Teflon, nylon, polyethersulfone, or it can be an FDA-approved cellulose cartridge having an equivalent pore size of about one micron. The presently preferred filter is an FDA-approved high efficiency pleated polypropylene cartridge available from Cole Palmer under part number A29830-20.

The filtrate is a concentrated solution of processed nicotine, which includes between about 8% and about 12% nicotine by weight. The concentrated solution of nicotine is added to enough purified water to produce a beverage which contains between about three mg and about four mg of nicotine in 250 ml of water. The beverage also includes 125 mg of potassium benzoate as a preservative, and 0.4 ml of lemon flavoring in each unit of 250 ml of beverage. Other preservatives, either natural or artificial, can be used with or as a substitute for potassium benzoate.

In the process just described, the heated mixture is cooled before filtering to protect the physical integrity of the filtering medium. However, other filters such as stainless steel screens can be used so that the mixture can be filtered while hot, if desired. In general, filtration is done at a pressure between about 5 and about 15 psi to force the liquid through the filter medium.

Seven people who each suffer from chronic migraine used the beverage prepared as just described to alleviate the symptoms and pains associated with the migraine attack. At the first symptom of the onset of a chronic migraine each subject drank eight ounces of the beverage containing four mg of the nicotine processed as described above. The eight ounces of beverage were consumed over a period between about two and about four minutes. Within between about two and about five minutes after consuming the beverage, all symptoms and pains associated with the migraine in each case was reported as eliminated. On a follow up of the seven test subjects over a period of 14 days, the pain associated with chronic migraine did not return.

Further tests will be conducted with additional subjects in accordance with the following protocols:

### I. Prevention Treatment

Fifty patients who have a history of at least two migraine attacks per month over a six-month period are treated in a double-blind test over a 60-day test period with a 30-day follow up. The test treats migraine symptoms at the onset of a migraine attack. Each patient receives four ounces of a water solution to drink within one to two minutes. Thirty five of the 50 patients receive four ounces of beverage which contains three mg of nicotine processed as described just above, 62 mg of potassium benzoate, and 0.2 ml of lemon flavoring. The other 15 patients receive four ounces of a placebo beverage of purified water containing 62 mg of potassium benzoate and 0.4 ml of lemon flavoring.

Each patent is examined two to three minutes after consuming each four ounce dose of beverage to determine the degree of relief from the symptoms of migraine attack. If the symptoms are not completely alleviated in a patient, that patient receives a second dose of four ounces of the same beverage previously administered. No test patient receives more than eight ounces of beverage in 24 hours. During the test period and follow up, the patients are checked for any side effects which might be due to the tested beverage.

### II. Alleviation Treatment

Fifty patients who each have a history of at least two migraine attacks per month over a six-month period are treated in a double-blind test for the prevention of the symptoms of migraine using the beverages described just above.

Schedule A:

Seven of a first group of 25 patients receive the placebo, and 18 patients receive the beverage with the processed nicotine. Each patient drinks the four ounces of assigned beverage within one to two minutes three times a week, say each Monday, Wednesday, and Friday for eight weeks. Each patient is followed up for a 30 day period to monitor for the occurrence of any symptoms of migraine and for any side effects which may result from taking the product.

Schedule B:

Seven of a second group of 25 patients receive the placebo, and 18 patients receive the nicotine-containing beverage described just above two times a week for eight weeks, with a 30 day follow-up to monitor for the occurrence of any symptoms of migraine attack or side effects from taking the product.

In addition to administering nicotine in beverage form for the treatment or prevention of migraine symptoms, the nicotine can also be administered in the form of a nasal spray, tablets, gel caps, transdermal patches, gum, or by injection.

In any case, the use of the nicotine should be at a rate so that the blood plasma nicotine level of the subject reaches at least about one nanogram of nicotine per about one milliliter of blood. Blood plasma levels of between about two and about 7.5 nanograms per milliliter of blood are typical for one dose of about one to about four mg of nicotine per day. In general, the nicotine prepared in accordance with this invention should be administered to a patient at the rate of about 1 mg to about 50 mg per 24 hour period.

It should be kept in mind that nicotine is a toxic compound. Therefore, only the minimum amount required for relief from smoking addiction or migraine symptoms should be used. Sixty mg of nicotine absorbed in one dose is lethal for an adult. Fortunately, nicotine is metabolized fairly rapidly. For example, it has a half life of about 60 minutes for people with average metabolic capacity for the compound. At this rate, one mg of nicotine absorbed in the body dissipates to about 0.031 mg within six hours.

The product of this invention can also be mixed with vitamins, natural or artificial fruit flavoring, cola mix, and natural fruit juices to provide a variety of beverages with the benefits described above. The beverage can also be carbonated.

The product of this invention may also be used in alleviating the symptoms of attention deficit hyperactive disorder (ADHD), attention deficit disorder (ADD), Toureete's Syndrome, schizophrenia, Parkinson's Disease, Alzheimer's Disease, anxiety, and depression.

What is claimed is:

1. A method for treating a patient subject to migraine headaches, the method comprising administering to the patient an effective amount of nicotine.

2. A method according to claim 1 in which the nicotine is in water at a concentration between about 0.4 and about 15 mg of nicotine per 250 ml of solution.

3. A method according to claim 1 or 2 in which the nicotine is administered to the patient at a rate to cause the nicotine level of the patient to reach between about 1 and about 10 nanograms in a per milliliter of blood.

4. A method according to claim 1 in which the nicotine is administered in the form of a beverage, nasal spray, tablet, gel cap, transdermal patch, gum, or by injection.

5. A method according to claim 1 or 2 in which the total amount of nicotine administered to the patient per 24 hour period is between about 1 and about 50 mg

6. A process for preparing a beverage treated with nicotine, the process including the steps of:
a) stirring a mixture of nicotine and water at a temperature above about 40° C. for at least about one minute;
b) filtering the mixture; and
c) adding the filtrate from the mixture to additional water to produce the beverage.

7. A process for preparing a beverage treated with nicotine, the process including the steps of:
a) stirring a mixture of nicotine and water at a temperature above about 75° C. for at least one minute;
b) cooling the mixture to below about 50° C.;
c) filtering the cooled mixture; and
d) adding the filtrate from the mixture to additional water to produce the beverage.

8. A process according to claim 6 or 7 in which:
a) the water and nicotine mixture is stirred for at least five minutes at a temperature about 100° C.; and thereafter
b) cooled to about room temperature before filtering.

9. A process according to claim 6 or 7 in which the nicotine is in the form of substantially pure nicotine.

10. A process according to claim 7 in which the nicotine is in the form of nicotine polacrilex.

11. A process according to claim 6 or 7 in which the mixture is stirred and boiled at about 100° C. for at least about five minutes during the heating step.

12. A process according to claim 6 or 7 in which the mixture is stirred and boiled for at least about thirty minutes.

13. A process according to claim 6 or 7 in which the filtering step includes filtering the mixture through activated carbon.

14. A process according to claim 6 or 7 in which the concentration of the nicotine in the heated and stirred mixture is at least about 20 mg in 250 ml of water.

15. A process according to claim 6 or 7 in which the concentration of the nicotine in the beverage after mixing the filtrate with the additional water is between about 0.5 and 20 mg in 250 ml of water.

16. A process according to claim 6 or 7 in which the filtering step includes filtering the mixture through a permeable membrane having an effective pore size below about 1 micron.

17. A processing according to claim 16 which includes applying a pressure of more than about 5 pounds per square inch to force liquid from the mixture through the membrane.

18. A process according to claim 6 or 7 in which the concentration of the nicotine in the heated and stirred mixture is between about 8% and about 10% by weight.
